(19) United States
(12) Patent Application Publication
(10) Pub. No.: US 2005/0095273 A1
(43) Pub. Date: May 5, 2005

(54) Synthetic resin-made glove and the process thereof

(76) Inventors: Taizo Yano, Heikura (JP); Mao-Sheng Lee, Taipei (TW)

Correspondence Address: Lee, Mao-Sheng, P. O. Box 55-846, Taipei, 104, TW

(21) Appl. No.: 10/697,201

(22) Filed: Oct. 29, 2003

(51) Int. Cl. A61K 9/70

(52) U.S. Cl. 424/443

(57) ABSTRACT

A glove made of synthetic resin has collagen powder homogeneously dispersed and cohered on an inner surface of the glove shell with the outer portion of the collagen powder exposed on and outwardly protruded from the inner surface of the glove shell, thereby maximizing the water absorbing and releasing property of the collagen powder to impart the warm, gentle and comfortable wearing feel as worn by the user's hand and contacted by the user's hand skin, to maintain a proper humidity within the glove, to overcome any cold feeling in the glove interior especially in winter, and also to reduce the production cost of the glove.

### BACKGROUND OF THE INVENTION

A conventional synthetic resin-made glove may be formed by dipping a glove form (or former) in a vinyl chloride resin or acrylic resin sol which however is blended with much plasticizer. The plasticizer will be released during the glove forming or molding process to be sticky to thereby affect the comfortableness for wearing the glove and also increasing the difficulty when released from the glove form.

In order to increase the wearing or tearing convenience or to enhance the releasing property from the glove form, many conventional resinous compositions had been developed such as to provide a lubricating layer on the inner surface of the laminated glove shell. However, it may increase the production cost. Also, its wearing feel (level of comfort) or convenience for the user is still not good.

If the synthetic-resin glove is used as a disposable glove such as for domestic or medical applications, it should be convenient to use, comfortable to wear, hygienic to dispose and also low in cost.

U.S. Pat. No. 4,185,330 to phyllis H. Stager disclosed a disposable cosmetic glove including an outer glove shell of lotion impervious material and an inner glove lining of lotion absorbent material which may be uniformly impregnated with a cosmetic lotion. However, the inner location absorbent layer in addition to the outer shell may increase the total thickness of the glove to thereby affect its wearing comfortableness and also influencing its elongation or maneuverability when wearing such a glove for working or operation.

The present inventor has found the drawbacks of conventional gloves and invented the present glove made of synthetic resin.

### SUMMARY OF THE INVENTION

The object of the present invention is to provide a glove made of synthetic resin having collagen powder homogeneously dispersed and cohered on an inner surface of the glove shell with the outer portion of the collagen powder exposed on and protruding from the inner surface of the glove shell, thereby maximizing the water absorbing and releasing property of the collagen powder to impart a warm, gentle and comfortable feeling when worn by the user. Also, once in contact with the user's skin, it will maintain a proper humidity within the glove, overcoming the cold feeling in the glove interior especially in winter, and reducing the production cost of the glove.

### DETAILED DESCRIPTION

According to the present invention, the collagen powder is homogeneously dispersed and cohered on an inner surface of the glove shell and the collagen powder is exposed on an outer surface of the inner surface portion of the glove shell.

The collagen powder is homogeneously dispersed and cohered on the inner surface in the glove, thereby providing a leather-like soft smooth feeling as touched by the hand skin when felt from within the glove, and also making an improvement over the dry corrugated feeling when touching the surface of a conventional glove coated or formed with inorganic particles or hard resin layer on the inner surface of the conventional glove.

The collagen powder of the present invention has good water-absorbing and water-releasing property (or properties). When the hand wearing the glove is dry, the collagen powder on the inner surface of the glove shell will release moisture to humidify the wearer's hand to a proper humidity; while the wearer's hand sweats, the collagen powder in the glove will absorb the moisture to thereby keep a moderate humidity in the glove for comfortable wearing of the glove. The present invention will therefore regulate the humidity in the glove interior automatically.

The collagen powder is exposed on a surface layer of the inner surface portion of the glove shell so that the collagen powder will absorb or release the moisture in the glove interior to a maximum limit (effect). In other words, the humidity regulating speed as effected by the collagen powder will be accelerated by the glove of the present invention. In a cold weather, cold moisture in the glove interior will soon be absorbed by the collagen powder formed in situ in the glove. The wearer, once inserting his or her hand in the glove, he or she will never get an unpleasant cold feeling. Just integrally forming the collagen powder in the glove shell, without forming a thick lining in the glove shell like a conventional glove with laminated layers, the glove of the present invention may be produced at lower cost.

The collagen powder as homogeneously dispersed and cohered in the inner surface of the glove shell and the collagen powder as exposed on the surface layer of the inner (surface) portion of the glove shell of the present invention is present in a manner like a plurality of small islands dispersively standing in a sea with each island independently protruding upwardly above sea level. In this way, the particles of the collagen powder of the present invention are independently dispersed on the inner surface of the glove shell with each particle of the collagen powder protruding or exposed outwardly from the surface of the inner surface portion of the glove shell.

The collagen powder as “exposed” on the inner surface of the glove shell indicates a partial portion of each collagen particle is protruded outwardly from the surface of the inner surface portion of the glove shell to be exposed to the glove interior while confined by the inner surface of the glove shell, but the remaining portion or the “root” portion of each collagen particle is stably firmly cohered into the inner surface portion of the glove shell.

When forming the glove body of the present invention at a semi-gel state, the collagen powder is applied to be cohered on the inner surface of the glove shell. The particles of the collagen-powder as cohered on the inner surface portion of the glove shell will form a slightly corrugated layer on the inner surface of the glove and the synthetic resin will be impregnated into the corrugated layer of the collagen particles. When the resin is cured as heated, the collagen particles will be stably “implanted” in the inner surface of the glove shell like a boat anchoring in a port. The surface portion of the corrugated layer of the collagen particles, which is not covered or “buried” by the semi-gel resin, will be protruded outwardly from the inner surface of the glove shell (like the island protruding upwardly from the sea level) to be in contact with the wearer's hand when worn within the glove.

The glove may be produced in accordance with conventional dipping forming process by using synthetic resin of vinyl chloride or acrylic resin. The resinous sol of the synthetic resin may be prepared by adding additives including: plasticizer, vulcanization promoter, gelling accelerator, anti-oxidant, stabilizer, pigment, surface active agent, thickening agent, defoaming agent, dispersing agent, etc., into the synthetic resin of the present invention. A lubricating layer may be further laminated into the inner surface of the glove and then the collagen powder is cohered on the lubricating layer of the glove to form plural layers of the glove. However, a single-layer of glove shell is preferred in the present invention in consideration of simpler production process and lower production cost.

The collagen powder includes hydrophilic and hydrophobic type. However, considering the decay property and the deterioration in gelatine, the hydrophobic type collagen powder is preferred in accordance with the present invention.

Natural cow or pig leather is purified to produce collagen fibers, which are subjected to grafting copolymerization with the graft polymer as copolymerization from methylmethacrylic acid and glycyl-monomer to obtain collagen fiber bundle. The collagen fiber bundle is then pulverized to produce the collagen powder having an average particle diameter of 8-20 μm. If the average particle diameter is less than 8 μm, the collagen powder will provide poor water-absorbing and water-releasing property as well as poor absorption for absorbing the additives of the resinous sol composition. If the average particle diameter is larger than 20 μm, it will be difficult to homogeneously disperse the collagen powder into the inner surface of the glove shell. The collagen particle will protrude outwardly from the glove surface at an appreciable height to influence a smooth feel when contacted with the user's hand skin. Also, the coarse particles will be easily stripped from the glove surface.

The collagen powder will adsorb at least one additive as selected from the additives of anti-bacterial agent, fragrance agent and deodorant and will keep the additive as impregnated into the collagen powder for an appreciable time period in order to continuously impart the function of the additive. Such an additive may be selected from cationic surface active agent of high molecular weight, including: Flavonoid and poly phenols of tea extract.

The glove of the present invention may be made in accordance with the process comprising the following steps:
A. Dipping the glove form in the resin sol to cohere the resin sol on the surface of the glove form (or former);
B. Heating the glove form cohered with resin sol thereon to produce a semi-gel resin layer on the surface of the glove form;
C. Homogeneously cohering the collagen powder on the semi-gel resin layer on the glove form;
D. Re-heating the glove form to tightly cohere the collagen powder on the resin layer on the glove form to form a single-layer resinous film on the glove form; and then inverting and releasing the film from the glove form to make a glove integrally formed with collagen powder.

A synthetic resin glove made of vinyl chloride resin will be described hereinafter. A glove form made of metal or ceramic material is preheated to 50˜60° C. and then dipped into a resinous sol containing vinyl chloride resin and the proper amount of plasticizer to cohere the resinous sol on the surface of the glove form. The quantity as cohered on the glove form may be adjusted according to the sol tackiness and the dipping times. After dipping, the glove form cohered with resinous sol thereon is heated in a furnace at about 180° C. for 1.5˜2.5 minutes to form a semi-gel resinous layer.

The so-called “semi-gel” as defined in the present invention indicates a situation that the resinous layer on the glove form is not completely gelled and the resin will not gravitationally drop or drain from the surface of the glove form. Also, the resin surface still maintains a tacky and soft surface adapted for cohering the collagen powder when applied onto the resin surface.

The heating time and temperature to turn the resin sol into a semi-gel status depends upon the kinds of resin and the adding quantity of the plasticizer. The suitable indicators showing the semi-gel status include: checking the fluidity and gloss of the resin sol as adhered on the glove-form; and the stripping phenomena of the collagen powder which is cohered on the resin layer of the synthetic resin glove as produced.

By visual observation, the gravitational dropping of the resin sol from the glove form may be checked. The fluidity of the resin sol when heated may also be observed by checking whether there is a gravitational dropping of the resin sol from the finger tip of the glove.

The gelling step of the resin sol may also be usually observed by checking the gloss of the resin. If the resin sol cohered on the glove form still exists in a sol state, it will be glossy. However, the resin itself will absorb the plasticizer and gradually lose its gloss during the gelling step.

During the gelling step, the tackiness and softness on the surface of the semi-gel resin will be decreased to thereby weaken the anchoring effect of the collagen powder on the resin surface, namely, reducing the cohesion of the collagen powder on the resin layer. A simple test may be done to check the stripping phenomena of the collagen powder as stripped from the resin layer by placing a piece of adhesive tape on an inner surface of the glove at the frequent stretching or rubbing portion between the thumb and index finger in a time interval and under a pre-set pressure, and then the tape is removed to observe the stripping condition of the collagen powder as stripped from the resin layer to thereby determine the cohesion force between the collagen powder and the resin layer. By varying the heating temperature and time, a desired semi-gel result may be obtained. Accordingly, an optimum heating temperature and time period for obtaining a proper semi-gel resinous layer may be determined.

Several methods may be applied to cohere the collagen powder on the semi-gel resin layer of the glove, including: dipping the glove form in a fluidized trough by keeping the collagen powder floating in the fluidized trough to allow the collagen powder to be cohered on the glove form; or spraying coating on the glove form by spraying the collagen powder through a spray gun to cohere the collagen powder on the resin layer.

For homogeneously cohering the collagen powder on a complex-shape area of the resin layer, e.g., the area between neighboring fingers, the dipping of the glove form in a fluidized trough is preferred. Through the dipping step in the fluidized trough, the free collagen powder not cohered on the resin layer may be easily recovered and collected. In a bottom portion of the fluidized trough, an air-dispersing plate perforated with many fine holes through the plate is installed for delivering air or inert gas streamflow through the air-dispersing plate to float the collagen powder in the fluidized trough for cohering the resin layer on the glove form.

The collagen powder as frictionally deposited or weakly cohered on the resin layer may be removed from the resin layer of the synthetic resin glove by vibration or by air-purging. A conventional vibrator may be provided to vibrate the glove form to shake off the collagen powder not firmly cohered to the resin layer. Additionally, hot air at about 180° C. may also be applied to blow off the free collagen powder. Or, the vibration and hot air purging may be alternatively operated to remove the free collagen powder not cohered to the resin layer.

The glove form dipped with the semi-gel resinous layer is then heated at 180˜200° C. in a furnace for about 15˜20 minutes to cause the resin layer to be completely gelled and hot melted to form the resin film and for simultaneously cohere the collagen powder on the resin film. After cooling, the resin film is inverted and released from the glove form to obtain the synthetic resin glove of the present invention.

In order to provide medical or cosmetic functions, at least one additive of anti-bacteria agent, fragrant agent, deodorant or other additives may be incorporated into the collagen powder at a ratio of 0.1˜1.0% (by weight) based on 100% (by weight) of collagen powder. The additive may be pre-adsorbed into the collagen powder before being applied to the resin layer. Even when the resin film cohered with collagen powder is formed, the aforementioned additive may also be coated on the resin layer to be adsorbed by the collagen powder already cohered on the resin surface. The present invention will be described in detail in the following examples which are presented for explaining the present invention, but not to limit the present invention within such examples.

### EXAMPLE 1

Resin Sol:

A resinous sol of vinyl chloride is prepared to comprise the following ingredients (all in parts by weight):

| | |
|---|---|
| vinyl chloride resin | 100 |
| plasticizers | 90 |
| viscosity-reducing agent | 8 |
| stabilizer | 2.6 |
| filler | 3.0 |
| pigment | 4.8 |

The ingredients or additives as used in this invention are available in the market.

Collagen Powder:

The pulverized collagen powder is graft-copolymerized with graft polymer of methyl methacrylic acid and glycyl monomer to obtain hydrophobic collagen powder, having average particle diameter of 8 μm, oil absorption rate of 1.1 cc/g, and apparent bulk density of 0.31 g/cc.

Process for Making Synthetic Resin Glove:

The resin sol (as aforesaid in this Example 1) is kept at 40° C., and a glove form after being preheated to 50˜60° C. is dipped into the resin sol. The glove form dipped with the resin sol is then heated at 190˜200° C. in a furnace for 1.5 minutes to obtain a semi-gel resinous layer on the glove form.

The glove form with the semi-gel resin layer is then dipped in a fluidized trough having the collagen powder (as aforesaid in this Example 1) floating in the trough to homogeneously cohere the collagen powder on the surface of the resin layer. The free collagen powder not cohered on the resin layer is then shaken off from the glove form by a vibrator (with vibration amplitude 1.0 mm×frequency 1 c/s). A hot-air stream, at 190˜200° C. with flow rate of 0.5˜1.0 m³/sec and air pressure of 30˜45 mmAg, is applied to purge the glove form to blow off the free collagen powder not anchored on the resin layer.

The resin layer on the glove form is then heated at 180˜200° C. in a furnace for 13.5˜18.5 minutes to make the resin layer to be completely gelled to produce a resin film, which is cooled by natural air and released from the glove form to obtain a single-layer synthetic resin glove of the present invention.

### EXAMPLE 2

Example 1 is repeated, except that the resin sol is heated at 190˜200° C. in the furnace for 2 minutes to form the semi-gel resin layer. A single-layer glove of synthetic resin will also be made in this example.

### COMPARATIVE EXAMPLE 1

The glove form is dipped in the aforesaid resin sol and then heated at 180˜200° C. in a furnace for 15˜20 minutes to form a substrate film of the glove. A surface-treatment dipping solution is prepared by mixing 100 parts (by weight) of inner surface treatment agent for forming lubricating layer on an inner surface of the glove, the collagen powder 100 parts (by weight), and surface active agent. The glove form dipped with substrate film thereon is further dipped in said surface-treatment dipping solution. The collagen powder used in this comparative example is the same as that used in Example 1. However, when mixed in the dipping solution, the collagen powder may be “blended” into the dipping solution so that the collagen powder will be “buried” in the resin layer when finishing the glove.

Then, it is heated at 150˜160° C. in the furnace for 6˜7 minutes to form the lubricating resinous film containing collagen powder. After releasing the film from the glove form, a double-layer glove of synthetic resin containing collagen powder is obtained. The aforesaid inner surface treatment agent includes: resinous solid matter 9%; mica 2% and water 89%.

### COMPARATIVE EXAMPLE 2

Comparative Example 1 is repeated, except that, the inner surface treatment agent is not added with collagen powder and surface active agent. By the way, another double-layer glove of synthetic resin can also be made.

Evaluation of the Examples:

The evaluation result for discussing and comparing the test results of the above-mentioned examples and comparative examples is shown in Table 1 as hereinafter described. The following testing items are provided for checking the properties of the glove products as respectively made by the examples and the evaluation results are summarized in Table 1.

Friction Coefficient Inside the Glove:

A specimen is cut from the glove product of each example and each comparative example. The specimen is adhered on a glass testing plate (20 mm length×60 mm width×2 mm thickness) to reveal the inner surface of the glove as a top surface of the testing specimen. The testing plate is then secured on an angle-adjustable platform and a weight of 2 grams is loaded on the testing specimen. The platform is then tilted to vary its inclination angles from a horizontality to a verticality until the weight is sliding on the specimen. The inclination angle where the weight is starting to slide is designated as θs and the static friction coefficient μs can be obtained by:
μs=tan θs.
Water Absorbing Rate and Water Releasing Rate:

A specimen (90×95 mm) is cut from the glove product of each example and each comparative example and is placed in a thermostat room at a temperature of 22° C. and a relative humidity of 33% for 2 hours. The weight of each specimen is measured to be Wd₁ (g).

Then, each specimen is transferred into a thermostat equipment, wherein the temperature is kept at 35° C. and relative humidity is set as 85%, for 5 minutes and the specimen is then measured to obtain its weight to be Wh₅ (g). The specimen is continuously placed in such a thermostat equipment for 60 minutes and then measured to get its weight to be Wh₆₀ (g). Finally, each specimen is transferred to the thermostat room, wherein the temperature is adjusted to 23° C. and the relative humidity to 31%, for one hour and its weight is measured to be Wd₂ (g).

By calculation of the measured data, the following rates can be obtained:

$$\begin{matrix}Water Absorption Rate \\ after 5 minutes ( g / {m}^{2} )\end{matrix} = \frac{{Wh}_{5} - {Wd}_{1}}{0.09 \times 0.095}$$

$$\begin{matrix}Water Absorption Rate \\ after 5 minutes ( g / {m}^{2} )\end{matrix} = \frac{{Wh}_{60} - {Wd}_{1}}{0.09 \times 0.095}$$

$$Water Releasing Rate ( g / {m}^{2} ) = \frac{{Wh}_{60} - {Wd}_{2}}{0.09 \times 0.095}$$

Wearing and Removing Easiness of the Gloves:

Volunteers are invited to perform the test for wearing the glove and then removing the glove as made from the above-mentioned examples and comparative examples. The different degrees of easiness to wear or to remove the glove are indicated with the following marks each mark corresponding to a degree of easiness:
⊚: The glove can be worn or removed extremely easily.
◯: The glove can be worn or removed easily.
Δ: The glove is worn or removed difficultly.
X: The glove is worn or removed extremely difficultly.

The testing results are averaged and summarized in Table 1.

Feel of Wearing:

The feel for wearing the glove as made from each example is tested by the volunteers and the testing results are averaged and summarized in Table 1. The feel includes: cold feeling, adaptability, and operational convenience when wearing the gloves. The different degrees of feel are indicated with the following marks:
⊚: There is no cold feeling as sensed when wearing the glove initially; and the glove is compatible, gentle and comfortable to the wearer's hand, allowing a very free stretching movement of the fingers.
◯: A slight cold feeling is sensed whenever wearing the glove initially; and the glove is worn with a medium comfortableness and the fingers can still be stretched freely.
Δ: A cold feeling is sensed when wearing the glove initially;
and the glove is touched with a slight hard feel. The fingers are still stretchable.

X: A cold feeling is sensed just wearing the glove. The glove is worn uncomfortably. The wearer's hand is easily fatigued.

TABLE 1
Evaluation Result

| | | | Comparative | Comparative |
|---|---|---|---|---|
| Testing Items | Example 1 | Example 2 | Example 1 | Example 2 |
| Static friction | 0.325 | 0.353 | 0.384 | 0.480 |
| coefficient, μs | | | | |
| Water absorption | 0.770 | 0.620 | 0.304 | 0.140 |
| rate after 5 mins | | | | |
| (g/m²) | | | | |
| Water absorption | 1.135 | 0.959 | 0.538 | 0.339 |
| rate after 60 mins | | | | |
| (g/m²) | | | | |
| Water releasing rate | 0.936 | 0.749 | 0.421 | 0.246 |
| (g/m²) | | | | |
| Wearing and | ⊚ | ◯˜⊚ | ◯ | ◯ |
| removing easiness | | | | |
| Feel of wearing | ⊚ | ◯˜⊚ | ◯ | Δ˜◯ |

From the above-mentioned Table 1, the results of Examples 1 and 2 which are made in accordance with the present invention are better and improved than the comparative Examples 1 and 2.

For instance, the water absorption rate (after 5 minutes or after 60 minutes) and the water releasing rate, the present invention provides a better water absorption and releasing property, indicating a quick conditioning of the humidity in the glove interior for enhancing a comfortable wearing of the glove than other comparative examples.

Reviewing the water absorption rate after 5 minutes, the present invention shows a high absorption rate, causing a quick absorption of moisture existing in the glove interior and the reduction of moisture will overcome the cold feeling drawback especially in winter season.

Reviewing the much improved water absorption (e.g. after 60 minutes) and water releasing property as effected by the present invention, the humidity as influenced by the sweat within the glove will be buffered by absorbing water or releasing water by the collagen powder cohered on the inner surface of the glove of the present invention, thereby adjustably providing a proper humidity within the glove for comfortable wearing of the glove.

The lower friction coefficient of the present invention also provides a smooth, gentle and comfortable fit.

Even the glove of the present invention as described in Examples 1 and 2 is made of a single layer, the properties as effected by the present invention can still be better than that of the double-layer construction as given in the comparative examples. Therefore, the present invention may render a better stretchability for a more comfortable wearing and vivid maneuverability (dexterity).

All the particles of collagen powder are homogeneously protruded outwardly from the resin layer surface, like many small islands standing in the sea, not only for stably firmly “implanting” the collagen particle into the resin layer, but also for revealing the outer portion of each collagen particle to be in contact with the environment within the glove and with the wearer's hand for buffering the humidity in the glove interior for comfortable wearing of the glove. In comparison (Comparative Ex. 1 and 2), the collage powder is buried in the resin layer, not “exposed” on the inner glove surface like this invention, the results of the comparative Examples 1 & 2 are therefore inferior to the present invention.

It is not necessary for the present invention to further provide an inner lining for absorbing any medical or cosmetic lotion as disclosed in the prior art of U.S. Pat. No. 4,185,330. The collagen powder integrally formed in situ in the glove inner surface will make the glove thinner, easily stretchable, comfortable to wear, and inexpensive to produce.

1. A glove comprising a substrate layer made of synthetic resin having collagen powder homogeneously dispersed on and cohered to an inner surface portion of the substrate layer of synthetic resin of a glove shell; and said collagen powder forming a slightly corrugated surface on the substrate layer of the glove shell, with each collagen particle of the collagen powder anchored and cohered to the substrate layer of synthetic resin having an outer portion of said collagen particle protruding outwardly from said substrate layer into a glove interior within the glove to be contacted with the glove interior and with a wearer's hand when worn, whereby the collagen powder as formed on the glove shell will absorb or release moisture in the glove interior for buffering a humidity in the glove interior for comfortable wearing of the glove.

2. A glove according to claim 1, wherein said substrate layer of synthetic resin is formed as a single layer.

3. A glove according to claim 1, wherein said particle of said collagen powder has a particle size or particle diameter of 8˜20 μm.

4. A glove according to claim 1, wherein said collagen powder in said glove comprising at least an additive selected from the group consisting of: an anti-bacteria agent, a fragrant agent, and a deodorant.

5. A glove according to claim 1, wherein said collagen powder is a hydrophobic collagen powder.

6. A process for making a glove comprising the steps of:
A. dipping a glove form (or former) in a resin sol to cohere the resin sol on a surface of the glove form;
B. heating the glove form cohered with the resin sol to produce a semi-gel resin layer on the glove form;
C. homogeneously cohering the collagen powder containing a plurality of collagen particles on the semi-gel resin layer to form a slightly corrugated surface of the collagen particles on the resin layer to protrude an outer portion of each collagen particle outwardly from a surface of the resin layer;
D. re-heating the glove form to tightly cohere the collagen powder on the resin layer on the glove form to form a single-layer resinous film on the glove form; and inverting and releasing the resinous film from said glove form to obtain a glove of synthetic resin integrally formed with collagen powder therein.

7. A process according to claim 6, wherein the step for homogenously cohering the collagen powder on the semi-gel resin layer further comprising an auxiliary step for removing the collagen powder which is free and not cohered to the resin layer.

8. A process according to claim 7, wherein said auxiliary step for removing the free collagen powder comprising a vibration of the glove form to shake off the free collagen powder from the resin layer.

9. A process according to claim 7, wherein said auxiliary step for removing the free collagen powder comprising a purging by air or inert gas to blow off the free collagen powder from the resin layer.

10. A process according to claim 6, wherein the step for homogeneously cohering the collagen powder on the semi-gel resin layer comprising the placing of the glove form in a fluidized trough and floating the collagen powder in the fluidized trough for cohering the collagen powder on the semi-gel resin layer on the glove form.

11. A process according to claim 6, wherein said collagen powder has a particle size or diameter of 8˜20 μm.

12. A process according to claim 6, wherein said collagen powder is further added with an additive selected from: an anti-bacterial agent, a fragrance agent and a deodorant.

13. A process according to claim 6, wherein said collagen powder is a hydrophobic collagen powder by grafting copolymerization of collagen powder with a graft copolymer.
